(19) United States
(12) Patent Application Publication
(10) Pub. No.: US 2015/0335454 A1
(43) Pub. Date: Nov. 26, 2015

(54) Method For Non-Invasive Stand Alone High Efficiency Body Weight Loss And Reduction Of Food Intake

(71) Applicants: HARRY FARO TAJI, RALEIGH, NC (US); NADER AL-FAROUQI, RALEIGH, NC (US)

(72) Inventors: HARRY FARO TAJI, RALEIGH, NC (US); NADER AL-FAROUQI, RALEIGH, NC (US)

(21) Appl. No.: 14/283,969

(22) Filed: May 21, 2014

(51) Int. Cl. A61F 5/00

(52) U.S. Cl. CPC A61F 5/0089; A61F 5/003

(57) ABSTRACT

The present invention pertains to a method that effectively helps reducing body weight and food intake. The method includes self-administering and depositing into the stomach, via a capsule or similar carrier, various objects of various materials including an automatic self-inflating hygienic balloon or more, of various sizes, made of edible or non-harmful materials, by swallowing a capsule containing the balloon or the object, that self inflates to different pre-determined sizes, thus occupying a volume of stomach, hence reducing food intake and feeling of fullness. The method does not require any medical intervention, or inflation pumps and tubes . . . The capsule or the carrier can be self-employed in several sizes and made from Natural or non-harmful synthetics, wherein the inflation can be accomplished by utilizing natural materials, for both the balloon and its contents, preferably by Baking Soda and Vinegar, or Yeast based products.

### FIELD OF THE INVENTION

This invention relates to safe and cost effective methods that are easy and quick to self-employ by users for reducing weight, food intake and thus directly relates to the field of good health, by use of natural edible, and non-harmful materials at low cost. Accordingly, the invention relates to Public Health, by improving the general health of all users and effectively helps avoiding chronic diseases brought about by excessive weight that ultimately can lead to Cardiac and Diabetic and many other medical problems.

By employing this method of the invention, individuals, Health Care providers, governments' health organizations, acquire benefits by reducing general health problems caused by overweight. Such accomplishments reduce costs absorbed by public and private health services. Accomplishing these effects is a natural result of utilizing the methods of this invention, hence making life more enjoyable for users on a universal scale.

### BACKGROUND OF THE INVENTION

Obesity is becoming wide spread in modern times due to many reasons, but mostly to excessive intake of Food. There are hundreds and hundreds of claimed methods to reduce weight from herbal concoctions to physical workout, low intensity, high intensity, invasive medical or semi medical procedures using risky methods and non-edible or unnatural synthetic materials, and various medications. Because the main reason is food intake and because modern life presents new tasty products and the public is swayed by powerful advertising campaigns to enjoy such increasing quantity of tasty products, which are very fattening, obesity has become wide spread and it is now the cause of many health problems. High levels of Cholesterol, blocked arteries supplying blood to the Heart and Brains, Diabetic problems, Kidneys and many other health problems are caused by obesity and hence causing society to absorb increasing costs and hardships.

The need for a new effective and of reasonable cost method that can be employed promptly, safe, and easily available that controls and reduces food intake is in urgent demand. The object of this invention is to present the needed method which is new, unique, and certainly better solution that effectively and conveniently provides this needed method. All at a very reasonable cost, within the means of all.

To accomplish this it is necessary to reduce the space in the stomach which is mainly for receiving larger quantities of food, and therefore give a sense of fullness by allocate less room for food intake. Other similar methods attempt to accomplish this but are all cumbersome, classified as medical surgeries and invasive in nature, and on many occasions cause serious health complications and are very costly, making benefitting the public from such medical surgeries or procedures, limited to a small number of users.

There are numerous conventional solutions disclosed in the art for reducing weight, among these solutions there is provided a non-invasive procedure, with no justification for such claim, that uses procedures very much similar to clearly invasive procedures that are prone to accidents and complications. Such solution claimed as best so far, is truly a risky, cumbersome and time consuming, yet very costly affair. The procedure in this conventional solution requires mechanically inserting Balloons made from standard Plastics or PVC, to be inserted with inflating tubes connected to an air pump. The procedure will be repeated 30 days later for a second balloon then again 60 days later for the third balloon and finally another procedure is carried out to remove all three balloons by a set of tubes with mechanical clamps to remove the balloons. It is claimed to take 10 minutes to insert the balloon each time and about 20 minutes to take them out. A claimed 50 minutes of fairly risky medical work. Yet the reality as told by some users is that a total of about 90 minutes is often required. This is in addition to waiting time and travel time to and from the medical facility. Also several pre-tests and interviews will be made, usually over a period of several weeks until a person actually starts with this plan. The cost is monumental, and is usually beyond the means of most of those who need it.

Another conventional solution requires surgical intervention that reduces the size of the stomach and intestines to reduce food intake and size of stomach. Such surgical procedures, described briefly in the body of this clause, are of course invasive and can lead to many complications, and are always performed by qualified medical teams including doctors and carried out in special clinics and hospitals, or medical facilities. This solution is indeed complex and differs greatly from this invention which does away with all of this and can be done quickly and safely, at much less cost to the user.

In another conventional solution, balloons are inserted into the stomach by special tubes that are used to inflate the balloons by pumping air into the balloon and at a later stage—usually several months—are retrieved by inserting clamps connected to at the end of tubes and inserting this assembly into the stomach to retrieve the balloons. Again all of this is carried out by qualified Medical teams in a hospital or similar medical facility, at great cost and a high level of risk of several complications. Such invasive method is by far inferior to the method of this invention described herein rendering the invention a better and unique procedure and a new method at reduced costs and with many different parameters of application. In the United Kingdom an invasive balloon insertion procedure cost Pounds Sterling 2,600 equivalent to over $4,000. While we estimate a unit price realized in using this invention method can be around $100 or less.

This invention provides a method for reducing weight at reasonable cost yet non-invasive and quick to employ by the user himself, and avoid the complicated and costly interference of medical or specially trained staff.

### SUMMARY OF THE INVENTION

Among the objects of this invention is to provide a non-invasive method and product to be available on mass scale, to beneficiaries of weight reduction by reducing Food Intake at reasonable cost.

Another objective of this method is to avoid medical procedures and surgeries that involve serious health problems to users who seek weight loss by means of an inserted Balloon and inflating it after it is placed into the stomach—and replaces such process by simple self-administered product that is the Method and Invention described herein.

Another objective of this method is to avoid a second medical procedure to remove the Balloon and hence double the risk of complications that can be caused and has been caused to many in the past.

Another objective of this invention is to make the Balloon treatment for weight loss is a fast one that can be completed in a few minutes rather than days and perhaps weeks waiting for clinic appointments and repeating that at the end of the term specified mostly 6 months later.

Another object of this Invention is to make it possible for mass population to benefit from the method and improve health by loss of weight.

Another object of this invention is to give many alternatives of utilizing this method by designing the product for many Terms length such as 4 weeks to 16 weeks or longer periods, to suit many situations and depending on the severity and preference of the need for weight loss.

Another objective of this invention is to reduce the cost of the method a user is swayed to adopt from Dieting and special foods, to exercise at Gyms or suffering hunger pains. Joining a Gym team is quite costly as purchasing various capsules and Foods or any other methods used so far. This method must be the least costly and beneficial to large number of users.

### BRIEF DESCRIPTION OF THE DRAWINGS

The drawings enclosed are made as simple as possible with only main needed details. The drawings demonstrate the workings of this invention and method.

FIG. 1 illustrates a first stage of the method of the present invention showing the simple path of the Capsule to be swallowed that contains the special balloon. This is done by swallowing the capsule and is usually taken with a glass of water or juice or any other edible liquid.

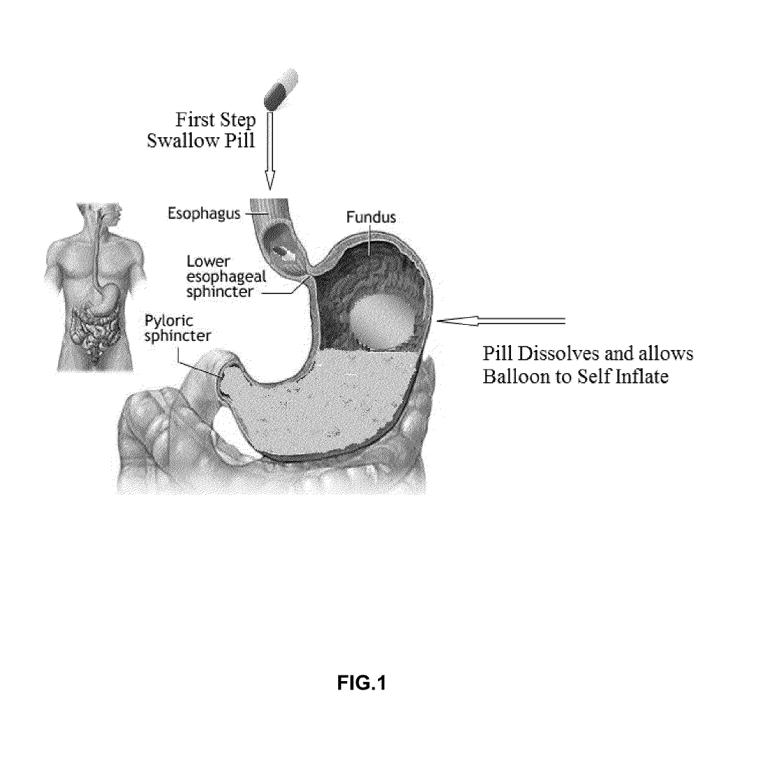

FIG. 2 illustrates a second stage of the method of the present invention showing the Balloon position in the stomach, floating and giving the user the feeling of fullness, thus reducing effect of appetite—while at the same time reducing the general size of the stomach. The balloon can be in this mode for the selected design duration.

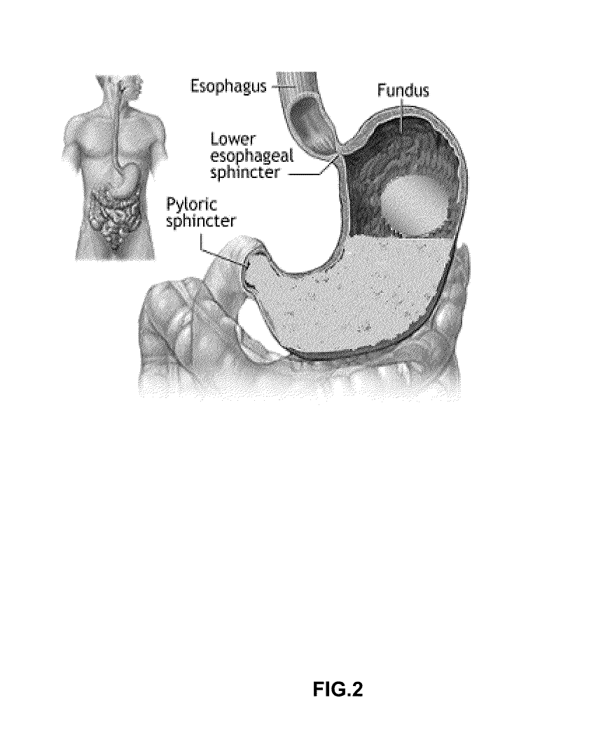

FIG. 3 illustrates a third Stage of the method of the present invention showing how the balloon breaks down and is digested and vacated from the digestive system at the end of the term selected.

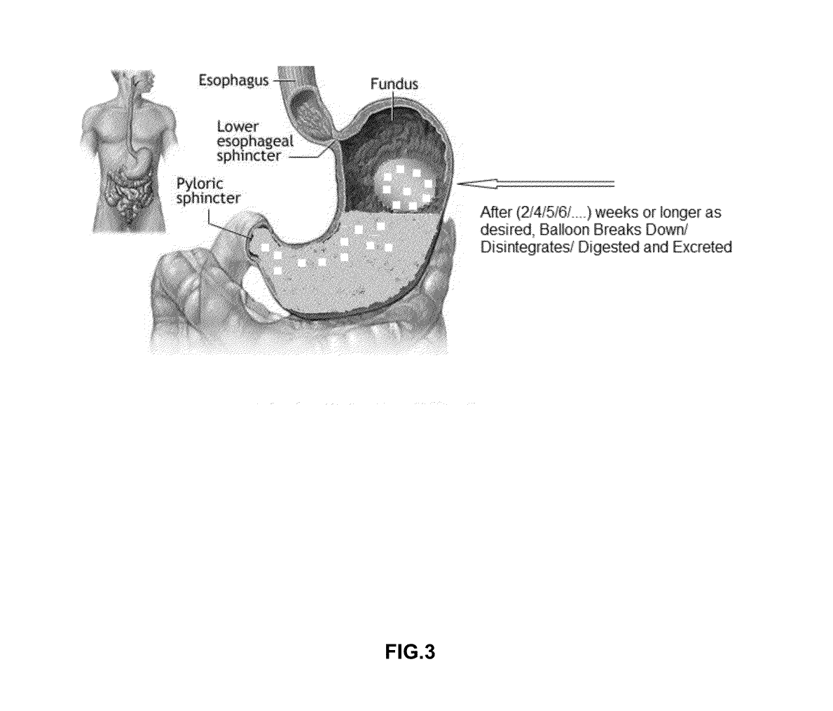

FIG. 4 illustrates two smaller balloons instead of the standard size Balloon, which can be used by persons who prefer to swallow smaller capsules. In all cases the larger capsule can be provided, in another form, with a small breathing tube on its' side that also dissolves after swallowing.

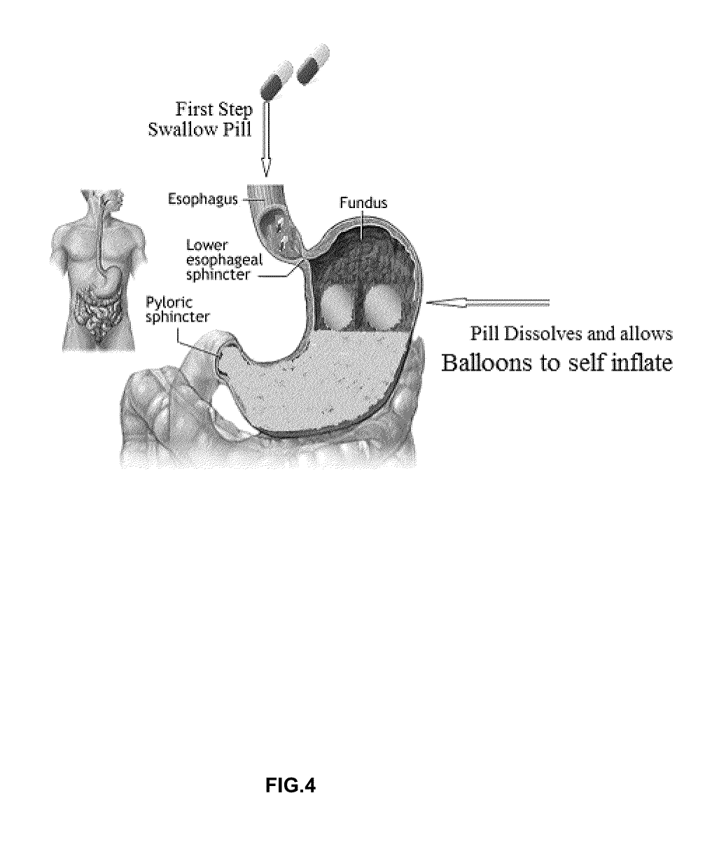

FIG. 5 illustrates a fourth stage of the method of the present invention showing how the capsule melts releasing the Balloon after being swallowed. Within minutes the Thread QD dissolves allowing the mixing of contents of both cavities Balloon sections. Within seconds the Balloon self inflates to designed size.

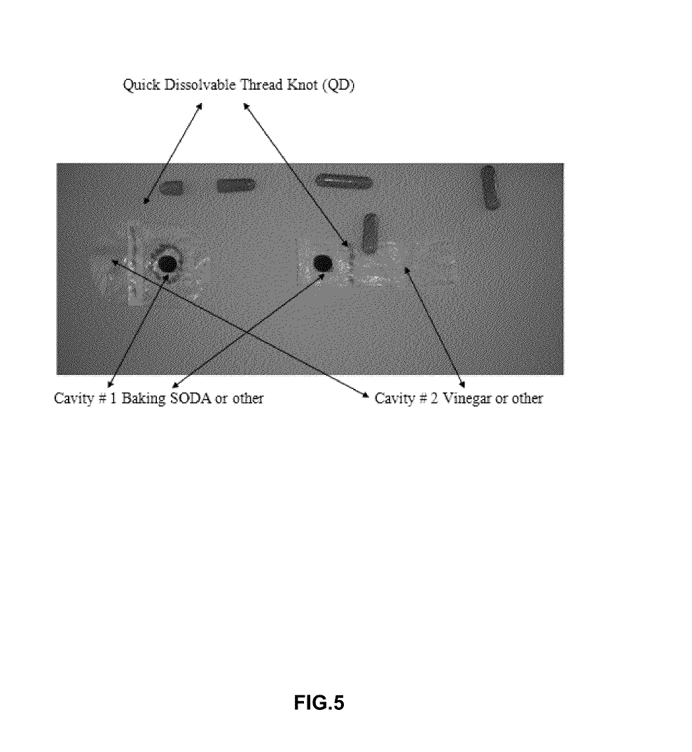

FIG. 6 illustrates balloons wrapped and inserted into the capsules.

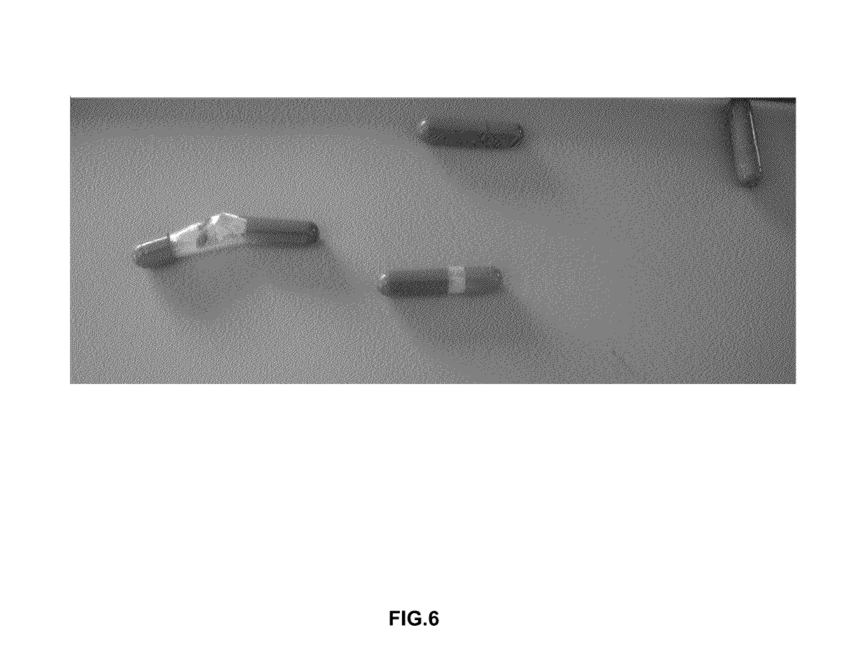

FIG. 7 illustrates balloons before sealing into the capsule.

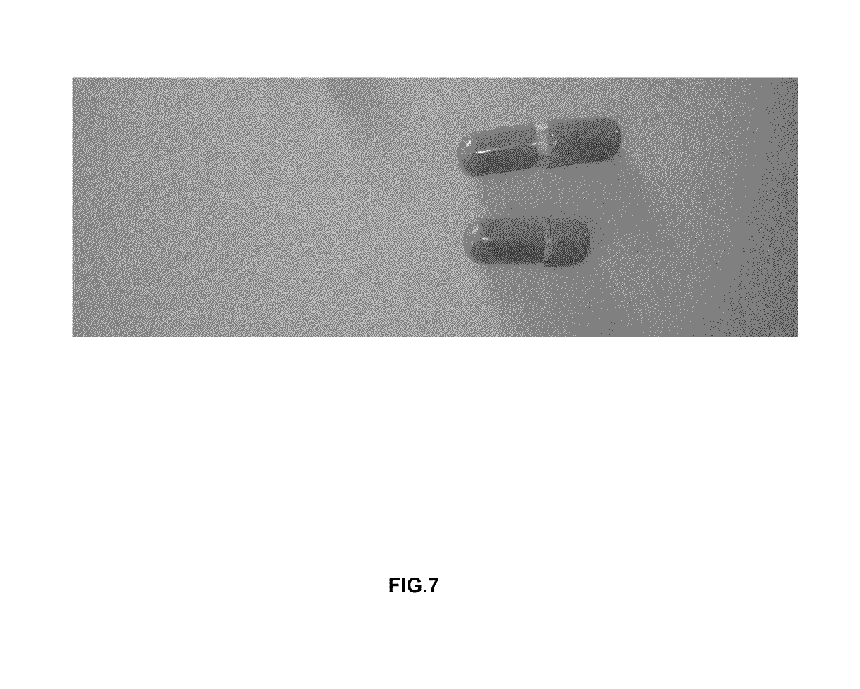

FIG. 8 illustrates sealed capsules after inserting balloons with contents for self-inflation

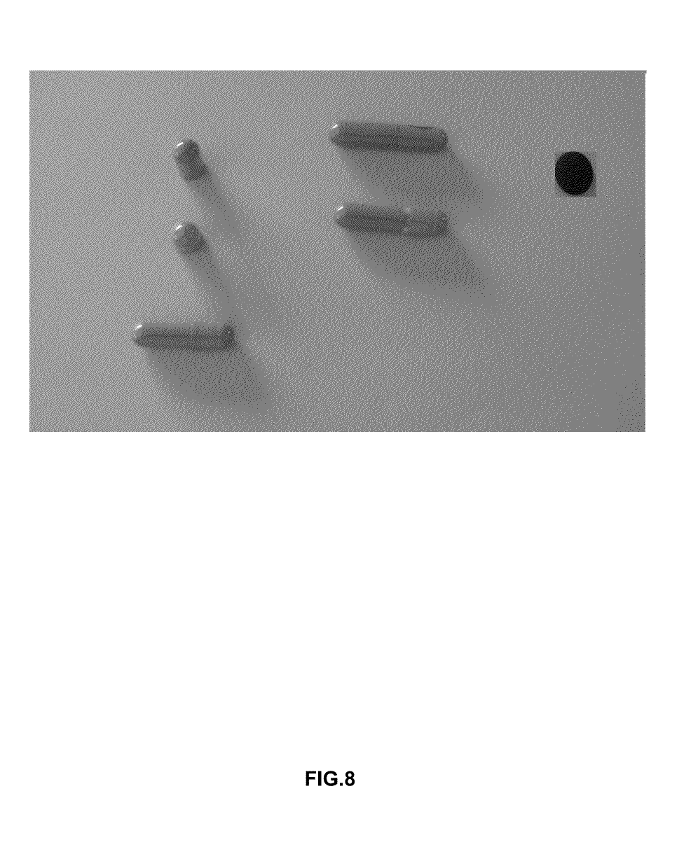

In the drawings the word Pill is used interchangeably with Capsule.

The drawings are expected to simplify envisioning the stages of the Method of this invention. Additional data is shown with each drawing to disclose fully the details of the method of this invention.

### DETAILED DESCRIPTION OF THE INVENTION

In one embodiment, the present invention provides a method and a product that is employed to Cause effective weight loss by users, and de-facto shrinkage of stomach size, by means of a non-invasive method that includes inserting a self-inflating balloon after it is swallowed in a capsule that dissolves in the stomach allowing the balloon to self-inflate.

The same can be accomplished by smaller size capsules with no breathing tube as they are easy to swallow. In such cases one or two such capsules with a smaller balloon inside can be used.

The Balloons are made from either non harmful synthetic materials or edible materials and of different thicknesses and composition to break down by several methods after various durations—terms. These Balloons can be made for dissolving and breaking down/disintegrating/digested after various selective various periods—term such as two weeks—4 weeks—5 weeks—six and longer, etc. as may be preferred and selected by users. Such balloons can also be made to breakdown or disintegrate (prematurely) any time the users decides by swallowing another capsule containing the needed degrading material of many compositions. This pre-term breakdown can also be accomplished by drinking a suitable liquid of various compositions or even some laxative products.

The Balloon can be made from Chitin or Chitosan, a very abundant material extracted mainly from shrimps' crust, and many other Crustaceans, insects and butterflies.

Such materials, known also as BIOPLASTICS, are available in huge amounts. Researcher Javier Fernandez, one of the founders of the Wyss Institute told CBS News that “the last 12 months they have produced the same amount of Chitin that

Equals all the plastics produced since 2009.

As such, the cost of the method of this invention can be maintained at affordable prices.

The self-Inflation process can be accomplished by packing inside the balloon via different manufacturing methods materials that cause Inflation to the designed size by themselves or by mixing the ingredients to cause the inflation, such as packing inside a yeast-based ingredient or two or more ingredients such as vinegar and baking soda, or other ingredients when mixed together.

The ingredients are separated inside the balloon by thin soluble or dissolvable thread that keeps them apart but later after the capsule is swallowed and the thin thread dissolves they are mixed and hence inflate the balloon without any interference by any mechanism of medical procedure or inserted tubes or other items to cause this inflation.

In another embodiment the inflation method can be accomplished by a compressed air cartridge that dissolves inside the balloon after it is swallowed and such causes inflation to the desired size.

The balloon can be made in different sizes such as small, medium, and large, and for different durations such as 2 weeks and gradually longer periods up to 6 or 16 weeks or more. As such more than 30 alternatives can be made available to select from.

One other important feature is the ability of the user to terminate this procedure any time after swallowing the Balloon, which can be accomplished by drinking some edible or synthetic liquids prepared for speeding up the breakdown and Disintegration of the Balloon when desired, or when the desired effects are attained earlier than anticipated.

After swallowing the Capsule, it melts releasing the Balloon. Within minutes the Thread QD dissolves allowing the mixing of contents of both cavities/Balloon sections. Within seconds the Balloon inflates to designed size.

We claim:

1- A method for non-invasive weight loss by swallowing a capsule containing a self-inflating balloon that stays in the stomach for various periods as selected by user.

2- The method of claim 1 is usable and beneficial to any overweight person.

3- The method of claim 1 can be employed by the user with no involvement of Medical staff or specially trained personnel.

4- The method of claim 1 can be employed anywhere without the need for a medical—clinical or specialized facility. It can be employed at home or at work.

5- The method of claim 1 wherein the user can swallow by means of a capsule containing the special self-inflating balloon with a drink of water or any other liquid or juice or the like.

6- The method of claim 1 wherein a balloon is packed in the capsule and swallowed to deposit it into the stomach.

7- The method of claim 1 where in the balloon is of special design and in many sizes when inflated by several means, such as mixing of two ingredients or more that are packed inside the Balloon and separated by a soluble knotted thread or other means that keeps the tow materials contained separate until the thread is dissolved and thus mixed together to cause inflation.

8- The method of claim 1 consists of a capsule that dissolves later after swallowing and containing the balloon filled to a pre-determined percentage of its volume with one or more ingredients that cause inflation after release from the dissolved capsule, that is swallowed to allow it to inflate.

9- The capsules and ingredients made from edible or synthetic non harmful ingredients.

10- The method of claim 1 consists of making the balloons from materials that degrade after the desired period of time, and made of—but not limited to—Edible materials, Chitin, Chitosan, Shrilk, or similar other materials.

11- The materials or ingredients used in this method are designed to disintegrate at pre-determined periods by various means such as selection of the materials they are made from or thicknesses or other means.

12- The method of this invention using the described capsule, balloons, and similar items swallowed for the purposes declared are designed so that they can be dissolved at any time desired by user by swallowing an ingredient that causes quick breakdown and disintegration of the balloon, whenever the user desires.
